(19) United States
(12) Patent Application Publication
(10) Pub. No.: US 2023/0309911 A1
(43) Pub. Date: Oct. 5, 2023

(54) PHONE CASE WITH SLIDABLE LENSES AND RELATED APPLICATION FOR IDENTIFYING SKIN CONDITIONS

(71) Applicant: L'Oreal, Paris (FR)

(72) Inventor: Brady Rowe, Clark, NJ (US)

(73) Assignee: L'Oreal, Paris (FR)

(21) Appl. No.: 17/710,040

(22) Filed: Mar. 31, 2022

(51) Int. Cl. A61B 5/00; G06T 7/00; G16H 20/00; H04M 1/02

(52) U.S. Cl. CPC A61B 5/441; G06T 7/0012; G16H 20/00; H04M 1/0264; G06T 2207/30088

(57) ABSTRACT

A system for detecting a skin condition including a case configured to hold a communication device, a support member attached to the case, a first lens slidably coupled to the support member and the case, a second lens slidably coupled to the support member and the case, wherein the second lens is configured to slide underneath the first lens, and wherein when an image is taken with the communication device, the first lens, the second lens, or the overlapped first and second lens modify the image, and an application on the communication device configured to detect a skin condition in the image.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a system for detecting a skin condition including a case configured to hold a communication device, a support member attached to the case, a first lens slidably coupled to the support member and the case, and a second lens slidably coupled to the support member and the case, wherein the second lens is configured to slide underneath the first lens, and wherein when an image is taken with the communication device, the first lens, the second lens, or the overlapped first and second lens modify the image, and an application on the communication device configured to detect a skin condition in the image is disclosed.

In another aspect, a method of detecting a skin condition including placing a communication device into a case, sliding a first lens, a second lens, or both lenses along a support member on the case to cover a camera of the communication device, taking an image with the camera of the communication device, and detecting a skin condition with an application on the communication device is disclosed.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an example system for detecting a skin condition, in accordance with the present technology;

FIG. 2 is another system for detecting a skin condition, in accordance with the present technology;

FIG. 3A is another system for detecting a skin condition, in accordance with the present technology;

FIG. 3B is an example system for detecting a skin condition in operation, in accordance with the present technology;

FIG. 4 is a flowchart of a method for detecting a skin condition, in accordance with the present technology.

### DETAILED DESCRIPTION

While illustrative embodiments have been illustrated and described, it will be appreciated that various changes can be made therein without departing from the spirit and scope of the invention.

Disclosed is a system for detecting a skin condition including a case, a communication device, and an application (“app”) on the communication device. The app is configured to work in conjunction with a case that includes one or more lenses configured to modify an image taken with the optical system (camera) of the communication device. In operation, a user may take an image (photo) of their skin with a communication device, with the one or more lenses covering the optical system of the communication device. In some embodiments, the app uses the modified image and/or AI to analyze the user's skin to identify skin conditions including, by not limited to, overall skin condition, acne, wrinkles, fine lines, scars, wounds, tumors, fungal infections, bacterial infections, viral infections, and dryness. In some embodiments, the app also assesses environmental conditions that can influence the state of the user's skin. Example conditions include, but are not limited to UV index, air quality, pollen, weather, temperature, and humidity. In some embodiments the app stores the image and the detected skin condition as a user's personal skin history. In some embodiments, the app also takes into account the user's personal skin history when detecting the skin condition. In some embodiments, the app includes a cloud platform. In some embodiments, the cloud platform can aggregate the images as a user's personal skin history.

In some embodiments, the app also assesses user inputs, including current skin condition, past skin conditions, past diagnoses (which can be both user inputs and past app diagnoses), and specific skin concerns. In some embodiments, the user can select specific skin concerns or conditions. In some embodiments, the user selection may include a questionnaire, giving the user a set of prompts to respond to in order to determine what skin condition they may have or what treatment may work best for treating said skin condition. In some embodiments, the questionnaire may help determine which of the lenses to slide over the camera when taking an image to detect the skin condition of interest.

In some embodiments, the app detects a skin condition. In some embodiments, the app detects a skin condition, and recommends a treatment. In some embodiments, the treatment is a cosmetic, a skin care product, a topical ointment or cream, an antifungal, an antibacterial treatment, an anti-viral treatment, and/or a recommendation to speak with a medical professional.

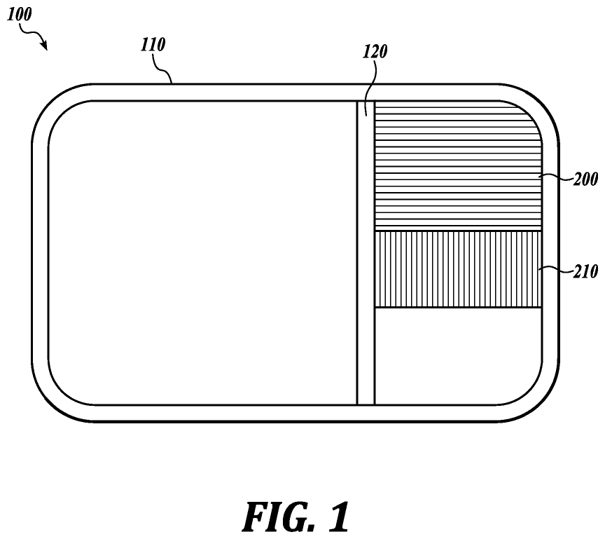

FIG. 1 is an example system for detecting a skin condition, in accordance with the present technology. In some embodiments, the system 100 includes a case 110, a support member 120, a first lens 200, and a second lens 210.

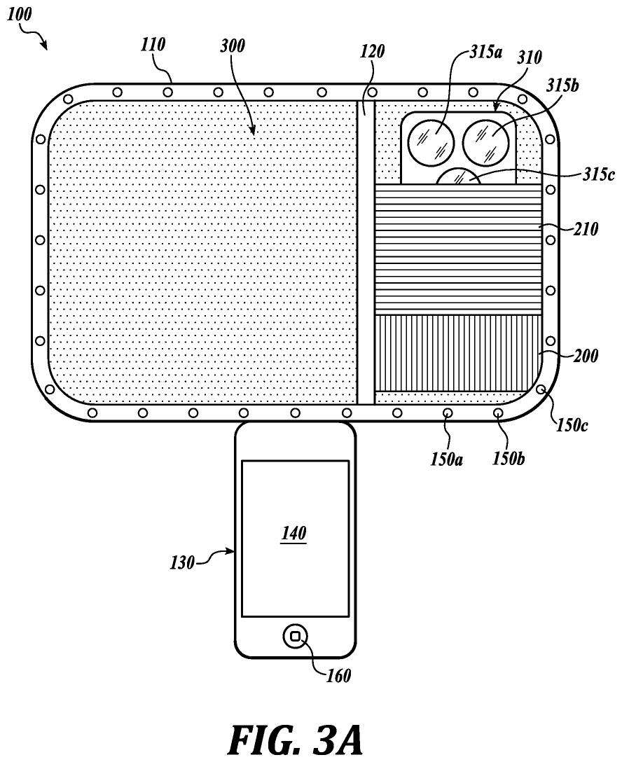

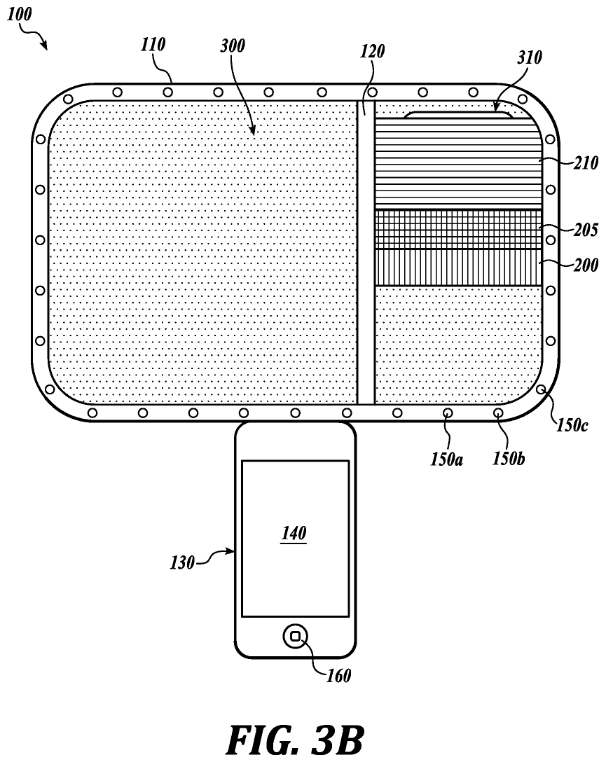

In some embodiments, the case is configured to hold a communication device (as shown in FIG. 3A-3B). In some embodiments, the case 110 is shaped to fit the communication device exactly, but in some embodiments, the case 110 may be thicker, or shaped in a way that extends the size or changes the shape of the communication device. In some embodiments, the case 110 is made of a flexible material, but in other embodiments, the case 110 may be rigid.

In some embodiments, the first lens 200 and the second lens 210 are slidably coupled to the case 110, so that the lenses 200, 210 can be slid along the length of the support member 120. In some embodiments, the case 110 includes a groove on a portion of the case 110 that holds the first lens 200 and the second lens 210. In some embodiments, the case 110 may have two grooves, so that the first lens 200 and the second lens 210 each have a separate groove. In operation, the lenses 200, 210 are slotted into the groove, disposed between the edge of the case 110 and the support member 120 so that they slide along the length of the support member 120. In some embodiments, the case is manufactured to that the lenses 200, 210 are already coupled to the case 110. In some embodiments, the lenses 200, 210 may be separate from the case 110, and can be inserted into the case by a user.

In some embodiments, the case 110 includes a support member 120. In some embodiments, the support member 120 extends along the entire width of the case 110, as shown in FIG. 1. In some embodiments, the support member 120 extends along the length of the case 110 horizontally. In some embodiments, the support member 120 has a groove to hold the first lens 200 and the second lens 210. In some embodiments, the support member 120 may have two grooves, so that the first lens 200 and the second lens 210 each have a separate groove. In operation, the lenses 200, 210 can be slid along the groove on the support member and the groove on the case to cover a camera of the communication device (as shown in FIG. 3B).

In some embodiments, the case 110 includes at least a first lens 200 and a second lens 210. While only two lenses 200, 210 are illustrated, any number of lenses can be incorporated onto the case 110. The first lens 200 and the second lens 210 may be any type of lens, including a parallel lens, a polarizer, a cross-polarizer, a linear polarizer, a circular polarizer, an elliptical polarizer, a colored lens, a magnification lens, and the like. In some embodiments, the first lens 200 is a parallel lens in a first orientation. In some embodiments, the second lens 210 is a parallel lens in a second orientation, perpendicular to the first orientation. In some embodiments, the first lens 200 is a vertical linear polarizer, and the second lens 210 is a horizontal linear polarizer. In operation, when the first lens 200 and the second lens 210 are layered on top of one another, the image is cross polarized. The first lens 200 and the second lens 210 may be any combination of lenses, for example, the first lens 200 may be a circular polarizer, and the second lens 210 may be another type of lens, such as a magnification lens. As such, in this example, when the first and second lens are layered on top of one another, the image is polarized and magnified. In some embodiments, the second lens 210 is configured to slide underneath the first lens 200.

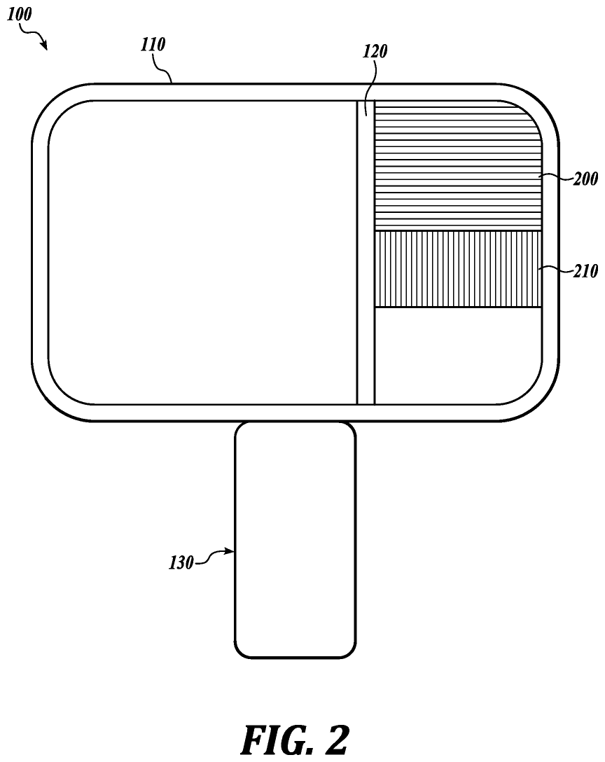

FIG. 2 is another system for detecting a skin condition, in accordance with the present technology. In some embodiments, the system 100 further includes a handle 130. While the handle 130 is illustrated similarly to a mirror handle in FIG. 2, the handle 130 may take any form, including a long support handle used to hold the camera at a distance from a user.

In operation, a user can hold the system 100 with the handle 130, and slide the first lens 200, the second lens 210, or both lenses 200, 210 over the camera of a communication device, and take an image of the skin, such as a face, arm, neck, or chest. In some embodiments, the user can take an image of a skin condition, such as acne, a wart, a scar, a wound or a raised bump on the skin.

FIG. 3A is another system 100 for detecting a skin condition, in accordance with the present technology. In some embodiments, the system 100 includes a power source 140, inside the handle 130, a plurality of lights 150a, 150b, 150c, along the edge of the case 110, and one or more actuators 160.

In some embodiments, the plurality of lights 150a, 150b, 150c are LED lights. In some embodiments, the plurality of lights 150a, 150b, 150c are UVA LED lights. Although the plurality of lights 150a, 150b, 150c are illustrated as being disposed around the entirety of case 110, in some embodiments, the plurality of lights 150a, 150b, 150c may be disposed only partially along the edge of the case, such as along only the top edge of the case, or only the top and bottom edge of the case. In some embodiments, a power source 140 is communicatively coupled to the plurality of lights 150a, 150b, 150c. In some embodiments, such as those illustrated in FIGS. 3A-3B, the power source 140 is located on or inside the handle 130, but in other embodiments, the power source 140 is on or inside the case 310 or may be inside the communication device 300 itself. In some embodiments, the power source 140 is communicatively coupled the actuator 160. The power source 140 may be a battery but can take the form of any suitable power source.

In some embodiments, the handle 130 of the system 100 includes an actuator 160. In some embodiments, the actuator 160 is a button, as illustrated in FIGS. 3A-3B, but in some embodiments, the actuator 160 is a switch, a capacitive touch-type button, a lever, or the like. In some embodiments, the actuator 160 is communicatively coupled to the communication device 300.

In operation, a user may actuate the actuator 160 to turn on the plurality of lights 150a, 150b, 150c, take an image with the communication device 300, or both. In some embodiments, a user may actuate the actuator a first time to turn on the plurality of lights 150a, 150b, 150c, and then actuate the actuator a second time to take the image with the communication device 300. In some embodiments, the user may actuate the actuator a first time to turn on the lights 150a, 150b, 150c, and then actuate the actuator a second time to turn off the lights 150a, 150b, 150c. While the actuator 160 is illustrated as on the handle 130 of the system 100, in some embodiments, the actuator 160 may be on the case 110 or on the communication device 300 itself.

In some embodiments, the system 100 further includes a communication device 300. In some embodiments, the communication device includes an optical system 310 including a plurality of cameras 315a, 315b, 315c. The communication device 300 is illustrated as a smartphone in FIGS. 3A and 3B, but the communication device may take any number of forms, including a tablet, laptop screen, desktop monitor, smart watch, and the like.

While the optical system 310 is illustrated as having three cameras 315a, 315b, and 315c, the communication device 300 may have any number of cameras. Further, while the optical system 310 is illustrated as being square and disposed on a right-hand side of the back of the communication device 300, the optical system 310 may take any shape or configuration. For example, the optical system 310 may be on the front of the communication device 300, i.e., on the same side as a screen of the communication device 300, so that the user may visualize the image before taking it with the system 100. In some embodiments, the optical system 310 is a single camera 315.

FIG. 3B is an example system 100 for detecting a skin condition, in operation, in accordance with the present technology. In some embodiments, the first lens 200, the second lens 210, or an overlap 205 of both lenses cover the optical system 310 before an image is taken.

In some embodiments, the first lens 200 and the second lens 210 are configured to modify the image taken by the optical system 310 of the communication device 300. In operation, the first lens 200, the second lens 210, or the overlap 205 of both lenses are slid over optical system 310 of the communication device 300. In some embodiments, the first lens 200 modifies the image in a first way. In some embodiments, the second lens 210 modifies the image in a second way. In some embodiments, the overlap 205 of the lenses 200, 210 modifies the image in a third, distinct way. In some embodiments, there may be more than two lenses. In some embodiments, the overlap 205 of the two or more lenses provide even more image modifications. In some embodiments, the lenses 200, 210 are the same size. While the lenses 200, 210 are shown to overlap 205 only partially in FIG. 3B, the lenses 200, 210 may completely overlap over the optical system 310 of the communication device, so that all the cameras 315a, 315b, 315c of the optical system 310 are covered by both lenses 200, 210.

The first lens 200 and the second lens 210 may be any type of lens, including a parallel lens, a polarizer, a cross-polarizer, a colored lens, a magnification lens, and the like. In some embodiments, the first lens 200 is a parallel lens in a first orientation. In some embodiments, the second lens 210 is a parallel lens in a second orientation, perpendicular to the first orientation. In some embodiments, the first lens 200 is a vertical linear polarizer, and the second lens 210 is a horizontal linear polarizer. In operation, when the first lens 200 and the second lens 210 are layered on top of one another, the image is cross polarized.

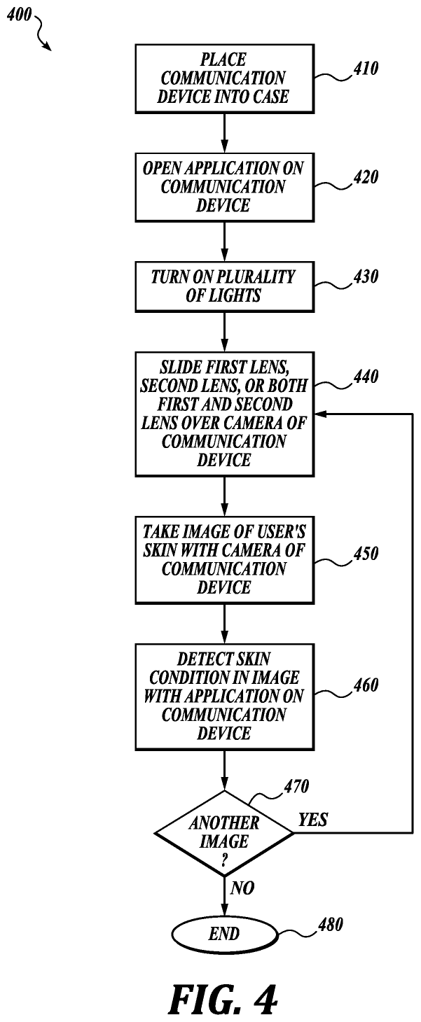

FIG. 4 is a flowchart representing an example method for detecting a skin condition, in accordance with the present technology.

In step 410, a user places a communication device into a case, such as case 110. In some embodiments, the communication device may be slid into the case. In some embodiments, the case is flexible, so the case may be stretched to accommodate the communication device. In some embodiments, the case is snapped onto the communication device.

Optionally, in step 420, the user can open an application (app) on the communication device. In some embodiments, step 420 occurs before step 410. In some embodiments, the app does not need to take the photo to analyze it, i.e., the photo may be uploaded into the app from another, separate application, such as a general camera application.

Optionally, in step 430, a plurality of lights (such as plurality of lights 150a, 150b, 150c in FIGS. 3A-3B) are turned on. In some embodiments, the plurality of lights are LED lights, or UVA LED lights. In some embodiments, the plurality of lights is located on the case. In some embodiments, the plurality of lights may be a separate component, not attached to the case. In some embodiments, the user can take the image without turning on the plurality of lights.

In step 440, the first lens, the second lens, or both lenses are slid over a camera of the communication device. In some embodiments, the first lens, second lens, or both lenses may be slid over more than one camera in an optical system, as shown in FIGS. 3A-3B. In some embodiments, the first lens modifies the image taken in step 450 in a first way. The second lens may modify the image taken in step 450 in a second way, and the overlapped first and second lens may modify the image in a third way.

In step 450, an image of the user's skin is taken with the camera of the communication device. In some embodiments, the image is of the user's face, but in other embodiments, the image can be any image of skin, including but not limited to an arm, a neck, a leg, a stomach, a shoulder, a back, or a skin condition of interest (such as a wrinkle, fine line, acne, blemish, lump, or infection). In some embodiments, the image may be one or more photos of the user's skin. In some embodiments, the image may be a 360-degree scan of the user's skin.

In step 460, an app on the communication device detects a skin condition in the image of the user's skin. In some embodiments, as described herein, the app uses the image modified by the one or more lenses, image processing, and/or AI to analyze the user's skin. In some embodiments, the app is configured to identify skin conditions including by not limited to overall skin condition, acne, wrinkles, fine lines, scars, wounds, tumors, fungal infections, bacterial infections, viral infections, and wounds. In some embodiments, the app also assesses environmental conditions that can influence the state of the user's skin. Example environmental conditions include, but are not limited to UV index, air quality, pollen, weather, temperature, and humidity. In some embodiments the app stores the image as a user's personal skin history. In some embodiments, the app also takes into account the user's personal skin history. In some embodiments, the app also assesses user inputs, including current skin condition, past skin conditions, past diagnoses (user inputs and/or past app diagnoses), and specific skin concerns. In some embodiments, the app includes a questionnaire to assess user inputs. In some embodiments, the user can select specific skin concerns or conditions from the questionnaire. In some embodiments, the questionnaire may include giving the user a set of prompts to respond to in order to determine what products would work best for their specific needs. In some embodiments, the app can alert the user to use a different lens (or combination of lenses) to better visualize the user's skin based on the responses to the questionnaire.

In some embodiments, the app detects a skin condition. In some embodiments, the app detects a skin condition, and recommends a treatment. In some embodiments, the treatment is a cosmetic, a skin care product, a topical ointment or cream, an antifungal, an antibacterial treatment, and anti-viral treatment, and/or a recommendation to speak with a medical professional.

In step 470, the user can take another image. In some embodiments, the user can take another image of the same portion of skin with a new lens, a new portion of skin, or an image with the lens configuration recommended by the app. If the user decides to take another image, the method 400 proceeds back to block 440. If the user decides to not take another image, the method 400 proceeds to block 480.

In block 480, the method ends.

1. A system for detecting a skin condition comprising:
a case configured to hold a communication device;
a support member attached to the case;
a first lens slidably coupled to the support member and the case; and
a second lens slidably coupled to the support member and the case, wherein the second lens is configured to slide underneath the first lens, and wherein when an image is taken with the communication device, the first lens, the second lens, or the overlapped first lens and second lens modify the image; and
an application on the communication device configured to detect a skin condition in the image.

2. The system of claim 1, wherein the case further comprises a handle.

3. The system of claim 1, wherein the case further comprises a plurality of LED lights.

4. The system of claim 3, wherein the plurality of LED lights are UVA LED lights.

5. The system of claim 3, wherein the handle includes a power source for the LED lights.

6. The system of claim 1, wherein the first lens is a parallel lens having a first orientation.

7. The system of claim 6, wherein the second lens is a parallel lens having a second orientation perpendicular to the first orientation of the first lens.

8. The system of claim 1, wherein:
the first lens is a vertical polarizer, and
the second lens is a horizontal polarizer.

9. The system of claim 8, wherein when the first lens and the second lens overlap, the image is cross polarized.

10. The system of claim 1, wherein the communication device is a smartphone.

11. The system of claim 1, wherein the skin condition is selected from a wrinkle, a fine line, acne, a scar, a tumor, a bacterial infection, a fungal infection, a viral infection, hyperpigmentation, or a wound.

12. A method of detecting a skin condition, with the device of claim 1, the method comprising:
placing a communication device into a case;
sliding a first lens, a second lens, or both lenses along a support member on the case to cover a camera of the communication device;
taking an image with the camera of the communication device; and
detecting a skin condition with an application on the communication device.

13. The method of claim 12, wherein the method further comprises lighting the user's face with a plurality of LED lights on the case.

14. The method of claim 12, wherein the skin condition is selected from a wrinkle, a fine line, acne, a scar, a tumor, a bacterial infection, a fungal infection, a viral infection, hyperpigmentation, or a wound.

15. The method of claim 12, wherein the application stores the image and the detected skin condition to generate historical data of a user's skin.

16. The method of claim 15, wherein the historical data of the user's skin is used to inform the detection of the skin condition.

17. The method of claim 12, wherein the application recommends a treatment based on the skin condition detected.

18. The method of claim 17, wherein the treatment is selected from a cosmetic, a skin care product, a topical ointment or cream, an antifungal treatment, an antibacterial treatment, and anti-viral treatment, or a recommendation to speak with a medical professional.

19. The method of claim 12, wherein the first lens is a vertical polarizer.

20. The method of claim 12, wherein the second lens is a horizontal polarizer.
